(19) United States
(12) Patent Application Publication
(10) Pub. No.: US 2014/0200405 A1
(43) Pub. Date: Jul. 17, 2014

(54) EXTENDABLE INTUBATION STYLET

(71) Applicant: RUTGERS, THE STATE UNIVERSITY OF NEW JERSEY, New Brunswick, NJ (US)

(72) Inventors: Glen Atlas, Livingston, NJ (US); Vikki Hazelwood, Wayne, NJ (US); Cailin Grunewald, New Windsor, NY (US); Tarik Kramcha, Denver, CO (US); Muhammad Abdul Rahman, Butterworth (MY); Chika Ekweghariri, East Orange, NJ (US)

(73) Assignee: RUTGERS, THE STATE UNIVERSITY OF NEW JERSEY, New Brunswick, NJ (US)

(21) Appl. No.: 14/076,876

(22) Filed: Nov. 11, 2013

Related U.S. Application Data
(60) Provisional application No. 61/726,931, filed on Nov. 15, 2012.

(51) Int. Cl. A61M 16/04; A61B 1/07; A61B 1/005; A61B 1/00; A61B 1/05; A61B 1/267

(52) U.S. Cl. CPC A61M 16/0488; A61B 1/05; A61B 1/267; A61B 1/0052; A61B 1/0011; A61B 1/07; A61M 16/0463; A61B 1/0057
USPC 600/109; 128/200.26; 600/249

(57) ABSTRACT

An extendable Intubation stylet including a handle member and a trigger mechanism moveable relative to the handle member between a retracted position and an extended position. A hollow semi-rigid stylet member extends from the handle member from a proximal end to a distal end. A flexible stylet member has a proximal end and a distal end and extends through the semi-rigid stylet member with the proximal end of the flexible stylet member connected with the trigger mechanism and configured such that when the trigger mechanism is in the retracted position, the flexible stylet member distal end is proximate the semi-rigid stylet member distal end and when the trigger mechanism is in the extended position, the flexible stylet member distal end is extended from the semi-rigid stylet member distal end.

### FIELD OF THE INVENTION

This invention relates to intubation stylets. More particularly, the invention relates to an intubation stylet with an extendible, flexible extension piece.

### BACKGROUND OF THE INVENTION

When a patient becomes apneic as a result of being anesthetized, an anesthesiologist must insert an endotracheal tube into their trachea to allow for the administration of oxygen as well as the elimination of carbon dioxide. Often, this is done in conjunction with video laryngoscopy. This is a procedure where the pharyngeal tissues are swept and compressed and a camera, at the end of the laryngoscope, allows for a view of the glottis.

Video laryngoscopy decreases time to intubation, diminishes cervical spine motion, and increases the chance of first pass success during difficult intubations. Video laryngoscopes are used in hospitals, ERs, and ambulances. Usage frequency and available equipment in these settings is expanding.

Video laryngoscopes are being used with increasing regularity in patients with known or anticipated difficult airways. While they usually provide excellent glottic visualization, directing an endotracheal tube, through the vocal cords, can be challenging. The laryngoscopy procedure is extremely cumbersome and requires both hands of the physician to intubate a patient.

Cases in which time is sensitive, the airway has been injured, or the airway is ‘difficult’ reveal problems with controlling the movement of currently commercially-available stylets. The difficult airway is one in which the physician experiences difficulty in securing an airway. Several anatomic and pathologic conditions have been identified that, if present, can reliably predict a difficult airway. Anatomic indicators include: obesity, large tongue, short neck, small jaw, and cervical immobility. Pathologic indicators include: blood, vomitus, airway edema, and fecial or neck trauma.

Difficult intubating conditions occur in 10.5%-18% of all intubations performed annually in the US. Failed intubations occur in 0.04%-0.07% of those cases. Regarding patients that require out of hospital emergency intubations, 7-10% of all intubations performed annually display difficult conditions. A difficult intubation can lead to decreased oxygen delivery to the brain, resulting in brain damage and even death. The cost of difficult intubations, including medical malpractice costs, surgical delays/cancellations, complications, and extended hospital stays, are estimated to be in the billions of dollars annually.

To begin the general intubation procedure, an anesthesiologist or emergency care professional would administer the anesthesia. After the patient is ‘under’, the procedure begins (although in some cases, due to time constraints, the procedure may begin before the patient is under). The doctor would stand at the head of the patient, with the patient lying on their back in front of them. The doctor holds the video laryngoscope in their left hand (regardless of their handedness); the endotracheal tube is placed over a stylet to help provide rigidity and guidance, and they are held in the right hand. First the doctor inserts the video laryngoscope, which compresses the tongue and (in non-difficult conditions) provides a view of the entry to the airway, the vocal cords. Then the doctor inserts the stylet, maneuvering past the epiglottis,

through the curved airway, through the vocal cords, into the trachea (roughly 1″ diameter). The doctor withdraws the laryngoscope to free their left hand, and advances the endotracheal tube down the trachea into place, while withdrawing the stylet from the tube. The doctor advances the tube a distance which is dependent upon the age and sex of the patient, using marking which are inked onto the tube in centimeters. Then the doctor inflates a small cuff near the end of the tube, inside the trachea, to prevent backflow and leakage of air, and starts pumping air into and out of the tube.

A major problem lies not in reaching the vocal cords and positioning the stylet outside of them, but in navigating past the vocal cords (an extremely delicate tissue) without touching them and in navigating through the airway without perforating the walls with the rigid stylets that are currently available.

### SUMMARY OF THE INVENTION

In at least one aspect, the present invention provides an extendable intubation stylet wherein the extendable endoscopic end of the stylet will allow for greater mobility and control when inserting the endotracheal tube past the epiglottis and into the trachea. The extendible stylet includes a flexible stylet member which is extendible relative to a semi-rigid stylet member.

In another aspect of the invention, the stylet may also incorporate a camera, for example a microchip camera, as well, as a light source, for example a fiber optic light source, which would allow the device to function as both a video laryngoscope and a stylet.

In another aspect of the invention, the degree of flexion of the flexible stylet member is controllable, for example, via Bowden cables in a hollow tube. This improvement could be used to create a motion similar to that seen with fiber optic laryngoscopes.

In yet another aspect of the invention, a suction port is incorporated which can be used to clear blood, and other secretions, from the pharynx. This would increase anatomic visibility when using the video laryngoscope.

In another aspect of the invention, the device handle may include a removable, disposable outer case-and extendable rod to reduce costs and risk of infection.

The extendable stylet expands the usage range of the video laryngoscope in various scenarios and creates a safer airway management method by filling the need for a softer, more maneuverable, and more flexible stylet. As a result, patients are provided with a safer alternative for tracheal intubation that limits the number of traumatic or failed intubations, tracheostomies, and morbidity when time is of critical value in saving lives.

The extendable stylet preferably simplifies the process of endotracheal intubation making it both safer and faster. The extendable stylet can be used in operative environments, ambulances, emergency rooms, intensive care facilities, as well as field use in military and trauma situations, and any other desired applications.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a side elevation view of an exemplary extendible stylet in accordance with an embodiment of the invention positioned within an exemplary endotracheal tube.

FIG. 2 is a perspective view of the extendible stylet of FIG. 1.

FIG. 3 is a perspective view of the handle member of the extendible stylet of FIG. 1.

FIG. 4 is a perspective view of the trigger mechanism and a portion of the flexible stylet member of the extendible stylet of FIG. 1.

FIG. 5 is a perspective view of the semi-rigid stylet member of the extendible stylet of FIG. 1.

FIG. 6 is a side elevation view; shown translucently, of an alternative exemplary stylet in accordance with the invention.

FIG. 7 is a cross-sectional view along line 7-7 of FIG. 6.

FIG. 8 is a cross-sectional view along line 8-8 of FIG. 6.

FIG. 9 is a side elevation view, shown translucently, of another alternative exemplary stylet in accordance with the invention.

FIG. 10 is a perspective view of another exemplary stylet in accordance with the invention.

FIG. 11 is a side elevation view, shown, translucently, of another alternative exemplary stylet in accordance with the invention.

FIG. 12 is a table showing A NOVA analysis between the rigid stylet and the extendable stylet of the present invention.

### DETAILED DESCRIPTION OF THE INVENTION

In the drawings, like numerals indicate like elements throughout. Certain terminology is used herein for convenience only and is not to be taken as a limitation on the present invention. The following describes preferred embodiments of the present invention. However, it should be understood, based on this disclosure, that the invention is not limited by the preferred embodiments described herein.

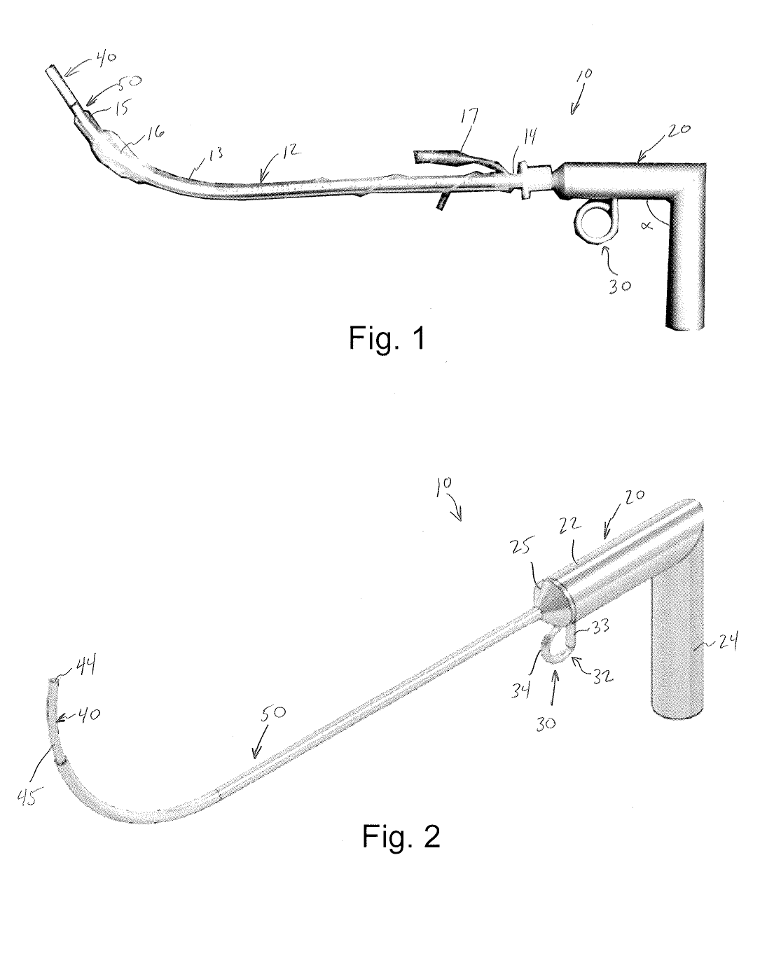

Referring to FIGS. 1-5, an exemplary embodiment of an extendible stylet 10 in accordance with an embodiment of the invention will be described. The extendible stylet 10 generally includes a handle member 20, a trigger mechanism 30, a flexible stylet member 40 and a semi-rigid stylet member 50. The flexible stylet, member 40 and the semi-rigid stylet member 50 are configured to be positioned within an endotracheal tube 12, as shown in FIG. 1, to facilitate placement of the tube 12 within a patient. The exemplary endotracheal tube illustrated in FIG. 1 includes a hollow shaft 13 extending from a proximal end 14 to a distal end 15. An inflatable cuff 16 is positioned adjacent the distal end 15 and is inflatable via an inflation lumen 17, as is known in the art. While an exemplary endotracheal tube 12 is illustrated and described, the invention is not limited to such and the extendible stylet 10 may be used with various devices.

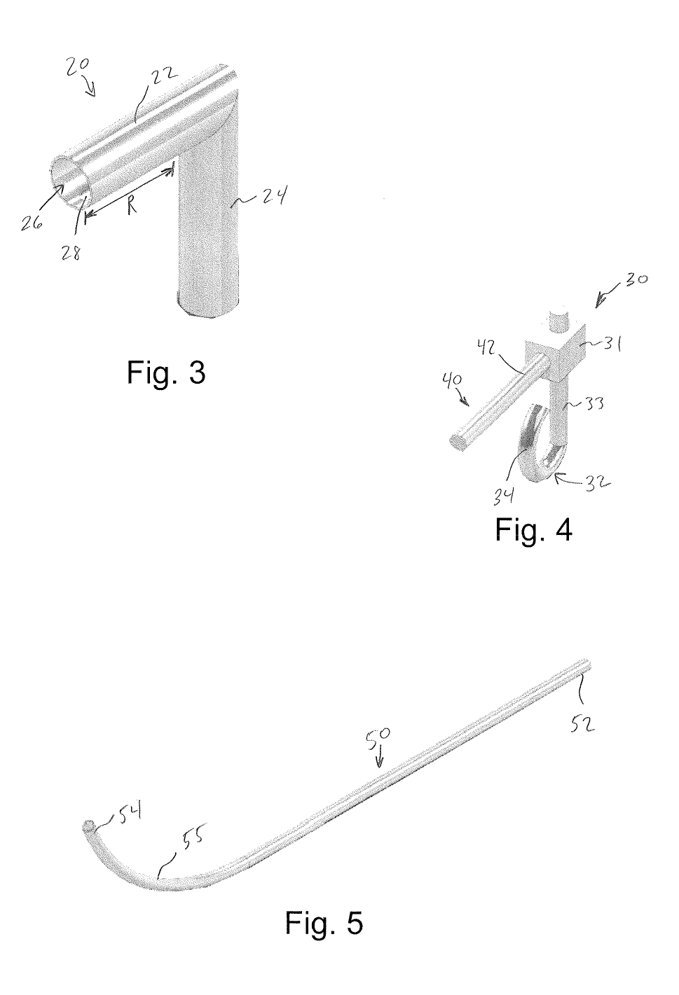

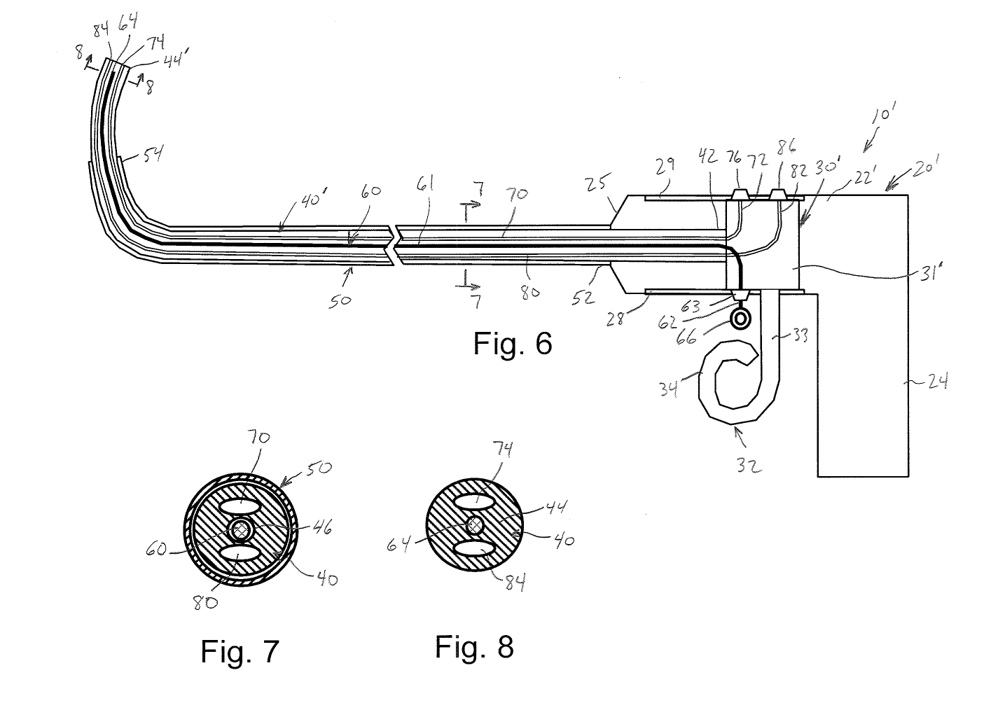

Referring to FIG. 3, the exemplary handle member 20 includes a hollow tube portion 22 having an open end 26. In the present embodiment, a grip handle portion 24 extends from the hollow tube portion 22, opposite the open end 26, at a 90° angle thereto. The handle member 20 is configured for holding in one hand by the user and may have different configurations, including but not limited to the configuration of the embodiment illustrated in FIG. 6. A guide slot 28 extends through the surface of the hollow tube portion 22 and is configured to receive a portion of the trigger mechanism 30 and guide movement thereof over a given range R. The handle member 20 is generally a rigid structure and may be made from stainless steel or any other desired material. It is contemplated that the handle member 20 may include a removable, disposable outer case and extendable rod (not shown) to reduce costs and risk of infection.

Referring to FIG. 4, the exemplary trigger mechanism 30 includes a slide block 31 configured to be received in the hollow tube portion 22 and to slidingly move therein. A trigger 32 is connected to the slide block 31 via a connection portion 33 which extends through the guide slot 28. As such, the trigger 32 extends into the acute angle a between the handle portions 22 and 24. The opposite end of the trigger 32 includes an engagement portion 34 which is positioned outside of the handle member 20. The illustrated engagement portion 34 has a loop configuration for engagement by a user's finger, however, other configurations may be utilized. As illustrated in FIG. 4, the proximal end 42 of the flexible stylet member 40 is connected to the slide block 31 such that movement of the slide block 31 via the trigger 32 will cause corresponding movement of the flexible stylet member 40. The slide block 31 preferably contacts the inside surface of the handle hollow portion 22 to provide a tactile feel, during extension of the flexible stylet member 40. Controlled movement of the slide block 31 via the trigger 32 allows precision movement of the flexible stylet member 40 and is not subject to sudden movement if the movement was controlled by a spring or the like.

Referring to FIG. 5, the semi-rigid stylet member 50 is a generally hollow tube extending from a proximal end 52 to a distal end 54. The distal end 54 is preferably manufactured with a predefined curved portion 55 proximate to the distal end. The semi-rigid stylet member 50 is preferably sufficiently rigid such that it maintains its shape even with an insertion force applied thereto and may be made from stainless steel or any other desired material. Referring to FIG. 2, the proximal end 52 of the semi-rigid stylet member 50 is preferably connected to a cap member 25 which closes the open end 26 of the handle member 20. The position of the semi-rigid stylet member 50 is preferably fixed relative to the handle member 20 at least daring use.

The flexible stylet member 40 extends through the semi-rigid stylet member 50 and preferably has a length equal to or slightly greater than the length of the semi-rigid stylet member 50 plus the length of the range of motion R. As such, when the trigger 32 is in the proximal most position of the range of motion R, the flexible stylet member 40 is withdrawn into the semi-rigid stylet member 50 such that the distal end 44 of the flexible stylet member 40 is proximate to the distal end 55 of the semi-rigid stylet member 50. This is preferably the initial position prior to use.

In use, the doctor positions the stylet 10 outside of the vocal cords, as with other stylets, but then instead of maneuvering the rigid stylet through the vocal cords, the doctor instead slides the trigger mechanism 30 distally to extend the flexible stylet member 40 through the vocal cords, into the trachea. The flexible stylet member 40 reduces the risk of damaging the sensitive vocal cord tissue or perforating the airway.

The flexible stylet member 40 is manufactured from a material having elastic or pseudo-elastic properties and has a rigidity less than that of the semi-rigid stylet member 50. Exemplary materials include nitinol (nickel titanium) alloy and low density polyethylene (LDPE), although other materials may be utilized. The material preferably has an elastic memory and the distal end 44 is preferably pre-formed with a curvature 45. In this way, when the flexible stylet member 40 is extended, the distal portion 44 will resume the curvature, In an exemplary embodiment, the flexible stylet member 40 is extended 2 inches while file curvature 45 has a radius of 3.4 inches. These dimensions are exemplary only and are not intended to limit the scope of the invention. In addition to being softer than traditional stylets, this additional degree of flexion allows the doctor to maneuver past the vocal cords at an angle in cases in which they cannot center the device perfectly to the opening in the vocal cords, and have to enter the airway diagonally.

In addition to the flexion, the trigger mechanism 30 is used to control direction, speed, and force of insertion of the endoscopic end of the flexible stylet member 40, while retaining the tactile feel that is preferred by doctors. After positioning of the endotracheal tube 12, the stylet 10 would then be withdrawn, as with other stylets.

Referring to FIGS. 6-8, an extendible stylet 10′ in accordance with another exemplary embodiment of the invention will be described. As in the previous embodiment, the stylet 10′ generally includes a handle member 20′, a trigger mechanism 30′, a flexible stylet member 40′ and a semi-rigid stylet member 50. The extendible stylet 10′ is similar to the previous embodiment and only the differences will be described herein.

In a first aspect, the stylet 10′ includes a secondary control mechanism 60 configured to control the angle of flexion of the distal tip 44′ of the flexible stylet member 40′. In the illustrated embodiment, the secondary control mechanism 60 includes a semi-rigid cable 61 which extends from a proximal end 62 to a distal, end 64. The cable 61 may be made from steel, stainless steel or any other material which is sufficiently flexible to flex with the flexible stylet member 40′ yet apply a push or pull force as described below.

At the proximal end 62, the cable 61 extends from the handle 20′ through a port 63 and is connected to a control handle 66. In the present embodiment, the port 63 is provided through the block 31 and is aligned to move with the block 31′ in the slot 28. In this way, tension in the cable 61 is not affected by movement of the trigger 32 and block 31′ during extension of the flexible stylet member 40′. The cable 61 extends through the block port 63 and through a hollow lumen 46 of the flexible stylet member 40′ (see FIG. 7) to the distal end 64 of the cable 61 which is embedded in the distal end 44′ of the flexible stylet member 40′ (see FIG. 8). As such, as the doctor pushes or pulls on the control handle 66, the cable 61 will push or pull on the distal end 44′ of the flexible stylet member 40′, thereby controlling the flexion angle thereof. This improvement would also create a motion similar to that seen with fiber optic laryngoscopes.

The stylet 10′ further includes a fiber optic light lumen 70 which extends from a proximal end 72 to a distal end 74. The light lumen 70 extends through the flexible stylet member 40′ such that the distal end 74 is positioned at the distal end 44′ of the flexible stylet member 40′ such that the light is provided at the tip of the flexible stylet member 40′. The proximal end 72 of the light lumen 70 extends to a port 76 in the block 31′. In this way, the light lumen 70 is not affected by movement of the trigger 32 and block 31′ during extension of the flexible stylet member 40′. The port 76 extends through a second slot 29 in the handle hollow portion 22′ and is connectable to a light source. The stylet 10′ may also incorporate a camera (not-shown), for example a microchip camera, which may be configured to operate with the light lumen 70 or independently thereof. The camera would allow the device to function as both a video laryngoscope and a stylet. The image signals from the camera may be transferred from the stylet 10′ via a wire (not shown) or through wireless transmission, for example, Bluetooth™ transmission, to a monitor, computer screen, tablet, smartphone or the like.

Additionally, the stylet 10′ includes a suction lumen 80 which extends from a proximal end 82 to a distal end 84. The suction lumen 80 extends through the flexible stylet member 40′ such that the distal end 84 is positioned at the distal, end 44′ of the flexible stylet member 40′ such that the suction is provided at the tip of the flexible stylet member 40′. The proximal end 82 of the suction lumen 80 extends to a port 86 in the block 31′. In this way, the suction lumen 80 is not affected by movement of the trigger 32 and block 31′ during extension of the flexible stylet member 40′. The port 86 extends through the second slot 29 in the handle hollow portion 22′ and is connectable to a suction source. The suction lumen 80 can be used to clear blood, and other secretions, from the pharynx. This would increase anatomic visibility when using the video laryngoscope.

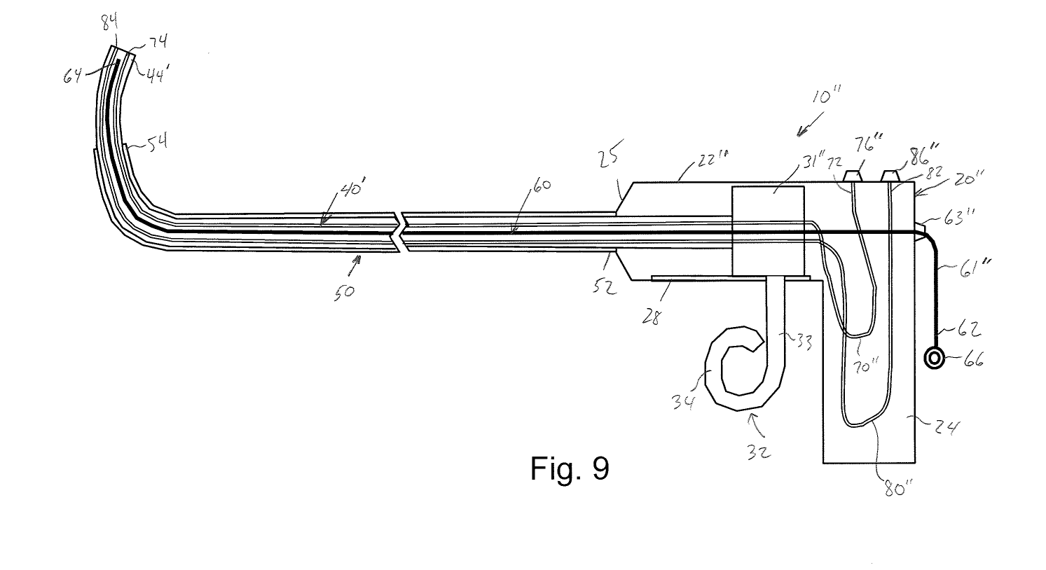

Referring to FIG. 9, an extendible stylet 10″ in accordance with another exemplary embodiment of the invention will be described. The extendible stylet 10″ is substantially the same as the extendible stylet 10′ except that the ports 63, 76 and 86 are not provided in the block 31″, hut instead the ports 63″, 76″ and 86″ are provided through the handle hollow portion 22″ of the handle 20″. The cable 61″, light lumen 70″ and suction lumen 80″ extend through passages (not shown) in the block 31″ or extend around the block 31″. To facilitate extension of the distal end 44′ of the flexible stylet member 40′, the cable 61″, light lumen 70″ and suction lumen 80″ are provided with additional length. In the illustrated embodiment, the additional length of the cable 61″ extends from the port 63″ while the additional length of the light lumen 70″ and the suction lumen 80″ extends within the grip handle portion 24. Positioning of the additional cable 61″ and lumens 70″, 80″ is not limited to the illustrated configurations, but may be otherwise configured, for example, through coiling. In other aspects, the stylet 10″ operates the same as the stylet 10′. While the stylets 10′ and 10″ are each described with a secondary control mechanism 60, a light lumen 70, 70″ and a suction lumen 80, 80″, these elements can be incorporated independent of one another and the inclusion of one does not require inclusion of each of the others.

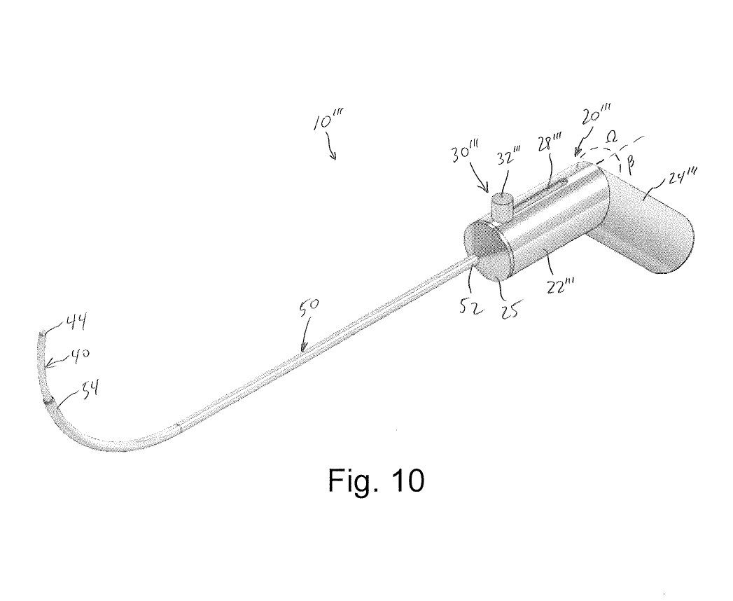

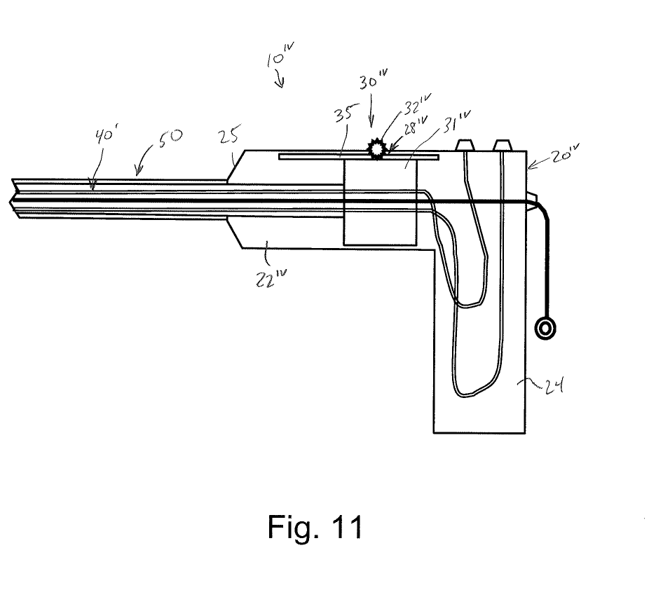

Referring to FIG. 10, another alternative exemplary extending stylet 10′″ will be described. The stylet 10′″ is substantially the same as the stylet 10 except in the configuration of the handle member 20′″ and the trigger mechanism 30′″. In the present embodiment, the grip handle portion 24′″ extends from the hollow tube portion 22′″ at an angle β less than 90°, for example at a 45° angle. Additionally, the guide slot 28′″ is on the opposite side of the hollow tube portion 22′″ and the trigger 32′″ of the trigger mechanism 30′″ is simply a nub extending out of the slot 28′″, In this way, the trigger 32 is located in the obtuse angle (Ω+β) between the handle portions 22′″ and 24′″. in this ease, the trigger mechanism 30′″ is controllable via the user's thumb. In all other ways, the stylet 10′″ is the same as the stylet 10 and operates in a similar manner.

Referring to FIG. 11, another alternative exemplary extending stylet 10^{iv} will be described. The stylet 10^{iv} is substantially the same as the stylet 10″ except in the configuration of the trigger mechanism 30^{iv}. In the present embodiment, the trigger mechanism 30^{iv} includes a trigger 32^{iv} in the form of a pinion extending through an opening 28^{iv} through the hollow tube portion 22^{iv}. The pinion 32^{iv} is rotationally supported relative to the handle member 20^{iv} and configured to engage one or more toothed racks 35 attached to the block 31^{iv}. Rotation of the pinion 32^{iv} causes linear motion of the rack 35 and thereby the block 31^{iv}. In this case, the trigger mechanism 30^{iv} is controllable via the user's thumb. In all other ways, the stylet 10^{iv} is the same as the stylet 10″ and operates in a similar manner.

Testing

Two test protocols, specified below, were constructed to compare the performance of the extendable stylet 10 to the rigid stylet. The criteria for each test originated from established successful and safe intubation standards. Intubations exceeding three attempts or 150 seconds in duration were considered to have failed.

1) Difficult Airway Test: The extendable stylet 10 was designed to decrease the number of failed intubations. Other available stylets are less efficient in maneuvering through Grade 3 airways (Cormack-Lehane classification). This test would simulate several difficult intubation conditions on mannequins, using this classification system. Success of the stylet would be based on intubation, success, number of attempts, and time.

2) ANOVA Test: By comparing the rigid stylet to the extendable stylet 10, the effectiveness of the extendable stylet 10 could be statistically proven, ANOVA analysis would be performed to demonstrate statistically significant differences between intubation successes, number of attempts at intubation, and intubation time.

Predicted Results

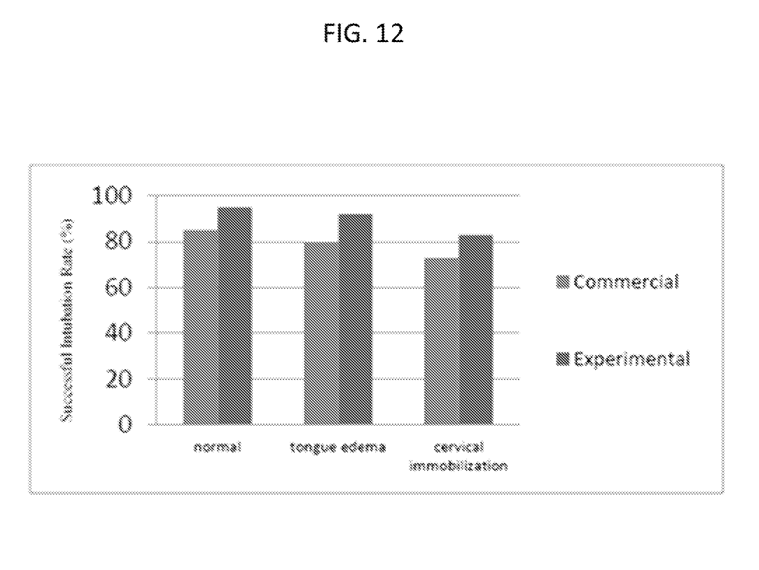

Results are pending. For the difficult airway test, intubations with the extendable stylet 10 are expected, to perform within a time limit of 150 seconds and within three attempts. The device is anticipated to demonstrate a success rate above 90% in multiple airway conditions. Optimally, the ANOVA analysis between the rigid stylet and the extendable stylet 10 would exhibit a statistically higher rating of successful intubations for the extendable stylet 10, as shown in FIG. 12.

The extendable intubation stylet is designed to improve the efficiency of tracheal intubations and to decrease the number of failed intubations. It is expected to provide more maneuverability, and greater control, than currently available stylets,

At the conclusion of the difficult airway test, intubations with the extendable stylet 10 are expected to have succeeded when used with the video laryngoscope. Furthermore, based on the results of the ANOVA test, the extendable stylet 10 is expected to be, at the minimum, statistically comparable to the rigid stylet.

These and other advantages of the present invention will be apparent to those skilled in the art from the foregoing specification. Accordingly, it will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention as defined in the claims.

What is claimed is:

1. An extendable intubation stylet comprising:
a handle member;
a trigger mechanism with a portion moveable relative to the handle member between a retracted position and an extended position;
a hollow semi-rigid stylet member extending from the handle member from a proximal end to a distal end; and
a flexible stylet member having a proximal end and a distal end, the flexible stylet member extending through the semi-rigid stylet member with the proximal end of the flexible stylet member connected with the trigger mechanism moveable portion and configured such that when the trigger mechanism moveable portion is in the retracted position, the flexible stylet member distal end is proximate the semi-rigid stylet member distal end and when the trigger mechanism moveable portion is in the extended position, the flexible stylet member distal end is extended from the semi-rigid stylet member distal end.

2. The extendable intubation stylet according to claim 1 wherein a portion of the trigger mechanism extends through a slot defined in the handle member, the slot defining a range of motion of the trigger mechanism.

3. The extendable intubation stylet according to claim 2 wherein the trigger mechanism includes a trigger which extends from the slot.

4. The extendable intubation stylet according to claim 3 wherein the handle member has first and second handle portions extending relative to one another with an acute angle therebetween and the trigger extends within the acute angle.

5. The extendable intubation stylet according to claim 4 wherein the trigger has a looped configuration.

6. The extendable intubation stylet according to claim 3 wherein the handle member has first and second handle portions extending relative to one another with an obtuse angle therebetween and the trigger extends within the obtuse angle.

7. The extendable intubation stylet according to claim 6 wherein the trigger has a nub configuration.

8. The extendable intubation stylet according to claim 6 wherein the trigger has a rotatable pinion configuration.

9. The extendable intubation stylet according to claim 2 wherein the semi-rigid stylet member has a first length and the flexible stylet member has a second length approximately equal to or slightly larger than the first length plus the length of the range of motion.

10. The extendable intubation stylet according to claim 1 wherein the handle member includes a hollow handle portion and the trigger mechanism moveable portion includes a block which is connected to the flexible stylet and is moveable within the hollow handle portion.

11. The extendable intubation stylet according to claim 10 wherein the trigger mechanism further includes a trigger extending from the block and out of a slot defined by the hollow handle portion.

12. The extendable intubation stylet according to claim 1 wherein the distal end of the semi-rigid stylet member includes a pre-defined curved portion.

13. The extendable intubation stylet according to claim 1 wherein the distal end of the flexible stylet member includes a pre-defined curvature.

14. The extendable intubation stylet according to claim 13 wherein the flexible stylet member is manufactured from a material having elastic memory such that the distal end of the flexible stylet member returns to the pre-defined curvature when the distal end is extended from the semi-rigid stylet member.

15. The extendable intubation stylet according to claim 1 wherein the flexible stylet member is manufactured from nitinol alloy or low density polyethylene.

16. The extendable intubation stylet according to claim 1 further comprising a secondary control mechanism configured to control an angle of flexion of the distal tip of the flexible stylet member.

17. The extendable intubation stylet according to claim 16 wherein the secondary control mechanism includes a cable extending through a hollow lumen of the flexible stylet member and having a distal end attached to the distal end of the flexible stylet member and a proximal end extending from the handle member and attached to a control member.

18. The extendable intubation stylet according to claim 17 wherein the cable proximal end extends from a port which is moveable with the trigger mechanism.

19. The extendable intubation stylet according to claim 1 further comprising a light lumen extending through the flexible stylet portion with a distal end of the light lumen positioned adjacent to the distal end of the flexible stylet portion and a proximal end of the light lumen extending to a port configured for connection to a light source.

20. The extendable intubation stylet according to claim 1 further comprising a suction lumen extending through the flexible stylet portion with a distal end of the suction lumen positioned adjacent to the distal end of the flexible stylet portion and a proximal end of the suction lumen extending to a port configured for connection to a suction source.

21. The extendable intubation stylet according to claim 1 further comprising a camera positioned adjacent to the distal end of the flexible stylet member.
